(19) United States
(12) Patent Application Publication
(10) Pub. No.: US 2008/0091022 A1
(43) Pub. Date: Apr. 17, 2008

(54) PREPARATION FOR METHSCOPOLAMINE TANNATE

(75) Inventors: Indravadan Ambalal Modi, Ahmedabad (IN); Keval Rameshkumar Sondagar, Ahmedabad (IN); Nirav Keshavlal Kagathara, Ahmedabad (IN); Manish Chandrakant Shukla, Ahmedabad (IN); Ravi Ponnaiah, Ahmedabad (IN); Bakulesh Mafatlal Khamar, Ahmedabad (IN)

Correspondence Address: DARBY & DARBY P.C., P.O. BOX 770, Church Street Station, New York, NY, 10008-0770, US

(73) Assignee: Cadila Phamaceuticals Ltd., Ahmedabad (IN)

(21) Appl. No.: 11/870,210

(22) Filed: Oct. 10, 2007

(30) Foreign Application Priority Data
Oct. 11, 2006 (IN) 1670/MUM/2006

(51) Int. Cl. C07D 491/08

(52) U.S. Cl. 546/91

(57) ABSTRACT

Methscopolamine Tannate is prepared from methscopolamine salt and tannic acid salt in aqueous medium. Methscopolamine salt is Methscopolamine bromide or nitrate, preferably methscopolamine bromide, whereas tannic acid salt is an alkali metal salt of tannic acid, preferably potassium salt of tannic acid. The preparation leads to methscopolamine tannate composition having a purity of about 93 wt. %, based on the weight of the composition, with the balance comprising primarily water.

### FIELD OF INVENTION

The invention relates to a process for preparing methscopolamine tannate composition having a purity of about 93 wt. %, based on the weight of the composition, with the balance comprising primarily water.

### BACKGROUND OF THE INVENTION

Antihistamines are available in the form of free bases as well as salts i.e. hydrochloride, maleate, tannate etc. Frequently it is necessary to utilize antihistamines in the form of tannate salt because such salts are generally quite stable and may be administered in such form without untoward side effects.

Tannic acid, also known as tannin, is a well-known naturally occurring substance. Commercially available tannic acid usually contains about 5 wt. % water, has a molecular weight of about 1702 and is typically produced from Turkish or Chinese nutgall.

Tannic Acid consists of an amorphous powder glistening scales or spongy masses varying in color from yellowish-white to light brown. Tannic acid is very soluble in water, glycerine or alcohol.

U.S. Pat. No. 6,790,980 B1 and U.S. Pat. No. 6,509,492 B1 describe various tannate compositions, which include methscopolamine tannate [Table-3 of U.S. Pat. No. 6,790,980 B1 and U.S. Pat. No. 6,509,492 B1], but do not give any details of preparing methscopolamine tannate.

As there is no published method available for preparing methscopolamine tannate, it is a long felt need for the industry to develop a commercially viable method for preparing methscopolamine tannate on an industrial scale.

### SUMMARY OF THE INVENTION

The main object of present invention is to provide a process for the preparation of methscopolamine tannate composition using commercially available raw materials.

Another object of the invention is to provide such a process, which is carried out in aqueous medium.

Yet another object of the invention is to provide a process for the preparation of methscopolamine tannate which results in the product of compositions having a minimum purity level of at least 90 wt. %, usually atleast 92.5% based on the weight of the composition.

The composition as prepared in accordance with the present invention contains about 7% (w/w) water of total weight of the methscopolamine tannate.

### DETAILED DESCRIPTION OF THE INVENTION

Methscopolamine tannate is different from other anti-histaminic tannates already in the market, in that its reactant is not a tertiary amine but a quaternary salt, and the chemistry involves, a replacement of an anion of quaternary methscopolammonium salt by a tannate anion generated from tannic acid using a base.

Thus in accordance with the present invention, methscopolamine tannate is prepared by following steps comprising,
(a) Reacting methscopolammonium salt with a salt of tannic acid in aqueous medium to give a slurry of solid mass,
(b) separating the compound by filtration/centrifugation, followed by washing with water and drying to give methscopolamine tannate.

The details of steps are elaborated as follows.

[A] Preparation of Methscopolammonium Salt Solution:

Methscopolammonium salt consist of quaternary ammonium cation with one of the anions such as bromide, nitrate and their like, capable of being replaced by tannic acid anion.

Methscopolammonium salt is dissolved in water.

The dissolution of methscopolammonium salt is carried out at 10 to 100° C., preferably at 20 to 40° C.

[B] Preparation of Tannic Acid Salt Solution:

Tannic acid salt is prepared by reaction of tannic acid with a base. The base is selected from C1-C4 alkoxides of alkali metals such as potassium, sodium or lithium. The preferred base is potassium tertiary butoxide. The preferred salt is Tannic acid potassium salt. Tannic acid salt is dissolved in water. The dissolution of tannic acid salt is carried out at 10 to 100° C., preferably at 20 to 40° C.

[C] Preparation of Methscopolamine Tannate:

To an aqueous solution of methscopolammonium salt, is charged, an aqueous solution of tannic acid salt, and stirred at 10 to 100° C., preferably at 20 to 40° C. more preferably at 25-30° C. to give slurry of solid mass. The compound is isolated by filtration, washed with water and dried to give methscopolamine tannate.

Alternatively, methscopolamine tannate is prepared by reversing the order of addition, wherein a solution of methscopolamine bromide is added to a solution of tannic acid salt in water.

Methscopolamine tannate on analysis found to contain 42% w/w to 48% w/w on as is basis of methscopolamine content and 43% w/w to 52% w/w on as is basis of tannic acid, % water is not more than 7%, residual solvent (here isopropyl alcohol) is not more than 2000 ppm. Description of methscopolamine tannate is pale yellow to greenish yellow powder.

The said invention of preparation of Methscopolamine tannate is further illustrated with examples, is set forth to aid in understanding the invention but is not intended to, and should not be construed to, limit the scope in any way.

### EXAMPLE-1

### Preparation of Methscopolamine Tannate

Stage-1 Preparation of K-Salt of Tannic Acid

In 1 L capacity multi necked flask equipped with condenser, water bath, thermometer, were charged tannic acid (100 gm), 500 ml of 2-propanol, stirred at 40-45° C. for about 15 minutes to get a clear solution.

A solution of potassium tert. butoxide (60 gms) in 2-propanol (600 ml) was added to it at 40-45° C. The reaction mass was stirred further for 2 hours at 40-45° C., cooled to room temperature and stirred for 30 minutes. The reaction mass was filtered and washed with 2×100 ml of 2-propanol and dried at 50° C. under vacuum to give 110-115 gms K-salt of tannic acid.

Stage-2 Preparation of Methscopolamine Tannate

In 5 L capacity multi necked flask equipped with, waterbath, thermometer were charged 3500 ml water and 100 gms of methscopolamine bromide and stirred at about 25-30° C. The reaction mixture became clear after about 10 minutes stirring at 25-30° C. A solution of K-salt of tannic acid (100 gm) in water (2000 ml) slowly added to it at 25-30° C. The reaction mass was further stirred for 30 minutes at 25-30° C., filtered and washed with 2×100 ml water. The wet cake was taken out and further stirred with 1000 ml water for about 30 minutes, filtered and washed with 2×100 ml water and dried at 45° C. under vacuum to give methscopolamine tannate

Wt=80.0 gms

### EXAMPLE-2

### Preparation of Methscopolamine Tannate

K-salt of tannic acid (60 gms) was dissolved in 750 ml water at 25-30° C. A solution of methscopolamine bromide (50 gm) in water (50 ml) is added at 25-30° C. during about one hour and further stirred for about 30 minutes. The reaction mass was filtered washed with 3×50 ml water and dried at 45-50° C.

Wt. of methscopolamine tannate =60 gms

We claim:

1. A process of preparing methscopolamine tannate comprising,
(a) Reacting methscopolammonium salt with a salt of tannic acid in water to give a slurry of solid mass,
(b) separating the compound by filtration/centrifugation, followed by washing with water and drying to give methscopolamine tannate.

2. A process of preparing methscopolamine tannate, as claimed in claim-1 wherein the reaction in step-(a) is carried out at 10 to 100° C.

3. A process of preparing methscopolamine tannate, as claimed in claim-2, wherein the reaction is preferably carried out at 20 to 40° C., more preferably at 25-30° C.

4. A process of preparing methscopolamine tannate, as claimed in claim-1 wherein the methscopolammonium salt in step-(a) is a compound wherein the associated anion is selected from Br, NO3.

5. A process of preparing methscopolamine tannate, as claimed in claim-4 wherein the preferred methscopolammonium salt is methscopolammonium bromide.

6. A process of preparing methscopolamine tannate, as claimed in claim-1 wherein the salt of tannic acid in step (a), is a compound wherein the associated cation is selected from alkali metal such as potassium, sodium, lithium.

7. A process of preparing methscopolamine tannate, as claimed in claim-1 wherein the preferred salt of tannic acid in step (a) is potassium salt of tannic acid.

8. A process of preparing methscopolamine tannate, as claimed in claim-1 wherein the medium of reaction in step-(a) is water.

9. A process of preparing methscopolamine tannate, as claimed in claim-1 wherein either an aqueous solution of methscopolamine salt is added to an aqueous solution of tannic acid salt or vice versa.
